(19) United States
(12) Patent Application Publication
(10) Pub. No.: US 2026/0021185 A1
(43) Pub. Date: Jan. 22, 2026

(54) LOCAL ANESTHETIC SOLUTION FOR DENTAL AND/OR CONTRAST MEDIA USE

(71) Applicant: Neocaine, LLC, Charlotte, NC (US)

(72) Inventor: Daniel Uzbelger Feldman, Charlotte, NC (US)

(21) Appl. No.: 19/345,876

(22) Filed: Sep. 30, 2025

Related U.S. Application Data
(63) Continuation of application No. 17/376,099, filed on Jul. 14, 2021, now abandoned.
Application No. 17/376,099 is a continuation of application No. 16/545,263, filed on Aug. 20, 2019, now abandoned.
Application No. 16/545,263 is a continuation of application No. 15/107,005, filed on Jun. 21, 2016, now abandoned.
(60) Provisional application No. 61/922,823, filed on Jan. 1, 2014.
(60) Provisional application No. 61/950,687, filed on Mar. 10, 2014.

(51) Int. Cl. A61K 47/26; A61B 6/00; A61B 6/03; A61B 6/51; A61K 9/00; A61K 9/08; A61K 31/137; A61K 31/167; A61K 31/245; A61K 31/381; A61K 31/445; A61K 45/06; A61K 47/02; A61K 47/12; A61K 47/18; A61K 47/20; A61K 49/04; A61M 5/00; A61M 5/14; A61M 5/178; A61M 11/00

(52) U.S. Cl. CPC A61K 47/26; A61B 6/032; A61B 6/481; A61B 6/485; A61B 6/51; A61K 9/0019; A61K 9/0043; A61K 9/0053; A61K 9/0063; A61K 9/08; A61K 31/137; A61K 31/167; A61K 31/245; A61K 31/381; A61K 31/445; A61K 45/06; A61K 47/02; A61K 47/12; A61K 47/183; A61K 47/20; A61K 49/04; A61M 5/007; A61M 5/14; A61M 5/178; A61M 11/00; A61M 2202/048; A61M 2210/0618; A61M 2210/0625

(57) ABSTRACT

An improved local anesthetic solution with diminished bitter taste includes an anesthetic agent, an anesthetic solution vehicle, and a bitterness suppressant. The bitterness suppressant includes one or more compounds selected from the group consisting of: a sugar selected from the group consisting of monosaccharide sugars, disaccharide sugars, polysaccharide sugars, and combinations of the any of the foregoing; sweet-tasting compounds; acids; amino acids; salts; miscellaneous suppressant substances; and combinations of any of the foregoing. The improved local anesthetic solution optionally includes one or more additional agents selected from the group consisting of: buffering agents; vasoconstrictors; preservative compounds; stabilizers; contrast media agents; and combinations of any of the foregoing.

### CROSS-REFERENCE TO RELATED APPLICATION

For purposes of the United States, the present application is a U.S. nonprovisional patent application of, and claims priority under 35 U.S.C. § 119 (c) to, each of U.S. provisional patent application Ser. No. 61/922,823, filed Jan. 1, 2014, which provisional patent application is incorporated by reference herein; and U.S. provisional patent application Ser. No. 61/950,687, filed Mar. 10, 2014, which provisional patent application is incorporated by reference herein.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

The present invention generally relates to improved local anesthetic solutions for dental and/or contrast media use.

Pain associated with dental treatment is typically managed through the injection of local anesthetics at the start of treatment. With the availability of a wide variety of local anesthetics and techniques, it is possible to achieve clinically adequate pain control in almost all situations.

In addition to block failure, one of the main drawbacks of local anesthetic injections in dentistry is the bad aftertaste associated with the bitterness of the cartridge solution composition. The active ingredients of local anesthetic solutions are generally responsible for the distasteful flavor of the drugs.

Bitterness not only refers to having or being a taste that is acrid and unpleasant, but also refers to sharpness of taste and/or an absence of sweetness. Many active pharmaceutical ingredients taste bitter and, thus, are commonly disliked by children as well as many adults.

Attempts to modify conventional local anesthetic formulations used in dentistry have been described through the addition of sugar alcohol and buffered solutions in order to increase anesthetic efficacy and decrease injection pain, respectively. For example, the use of sugar alcohol has been shown in U.S. Pat. No. 6,075,059 to Reader, and the buffered solutions approach for increasing the anesthetic pH has been set forth in U.S. Pat. No. 5,209,724 to Dhaliwal et al. and in U.S. Pat. No. 6,329,398 to Zappala.

In a recent abstract, Yangjie Wei, et al. proposed a cyclodextrine-based compound (hydroxypropyl-β-cyclodextrin) to prevent lidocaine hydrochloride from interacting with the taste receptors in the tongue (“Masking the Bitter Taste of Injectable Lidocaine HCl Formulation for Dental Procedures” AAPS 2013 Proceedings, R6144). Cyclodextrines are not considered sweeteners and are commonly used for solubilizing and stabilizing drugs. Additionally, cyclodextrines can be nephrotoxic when administered parenterally.

A recent suggestion for masking the bitter taste of dental anesthetic is disclosed in U.S. Patent Application Publication No. US 2014/0275170 A1 to Nehleber et al., whereby menthol and a cyclodextrine-based solvent are included in solution to mask the bitter taste. Menthol is an organic compound made synthetically or obtained from cornmint, peppermint or other mint oils. Menthol is also known to have a level of toxicity and can have a cardioaccelerating effect when ingested with foods. Furthermore, there exists a lack of regulatory approval for menthol with regard to injection purposes in humans.

With respect to the delivery mechanisms, a nasal spray for local dental anesthetic has been devised in U.S. Pat. No. 6,413,499 to Clay and in U.S. Pat. No. 8,580,282 to Kollar. Though research on the subject of nasal spray dental anesthetics has assessed anesthetic safety and efficacy, the bitterness effect of the spray is unexplored. In an assessment of two nasal sprays (azelastine nasal spray and triamcinolone acetonide nasal spray), testing determined that both medications were generally well tolerated. However, azelastine nasal spray led to more adverse events than triamcinolone acetonide nasal spray (56.9 and 19%, respectively; p=0.001) primarily as a result of the bitter taste of azelastine nasal spray.

Moreover, a needle-less system for dental local anesthetic purposes has been developed as per the U.S. Pat. No. 6,610,029 to Golan. Research in this area has demonstrated the effectiveness of the needle-less system technique without any focus on the clinical evaluation of the bitter aftertaste.

Furthermore, the mechanism of the prolonged effect of dextran on the duration of local anesthesia has been studied.

Since the development of the first commercial anesthetic, little effort has been dedicated to the improvement upon the bitter aftertaste common to local anesthetic solutions and nasal sprays.

A contrast medium is a substance used to enhance the contrast of structures or fluids within the body in connection with medical and dental imaging. Contrast media solutions have been advocated to be used for dental implant templates, caries detection, cracks and tooth fracture evaluation and periodontal pockets measurement, as well as for root canals irrigation and diagnostic assessment (as described in U.S. Pat. No. 5,106,301 to Miyahara et al. and U.S. Pat. No. 5,797,745 to Ruddle). Despite these efforts, anesthetic solutions and injectable cartridges have not been used as a vehicle for contrast media in the field of dentistry.

With this in mind, a need exists for improvement in local anesthetic solutions for use in treating pain. These, and other needs, are addressed by one or more aspects of the present invention.

### SUMMARY OF THE INVENTION

The present invention includes many aspects and features. Moreover, while many aspects and features relate to, and are described in, the context of improved local anesthetic solutions for dental and/or contrast media use, the present invention is not limited to use only in improved local anesthetic solutions for dental and/or contrast media use, as will become apparent from the following summaries and detailed descriptions of aspects, features, and one or more embodiments of the present invention.

Accordingly, one aspect of the present invention relates to an improved local anesthetic solution with diminished bitter taste. An exemplary such local anesthetic solution includes an anesthetic agent, an anesthetic solution vehicle, and a bitterness suppressant. Furthermore, in this aspect of the invention, the bitterness suppressant includes one or more compounds selected from the group consisting of: a sugar selected from the group consisting of monosaccharide sugars, disaccharide sugars, polysaccharide sugars, and combinations of the any of the foregoing; sweet-tasting compounds; acids; amino acids; salts; miscellaneous suppressant substances; and combinations of any of the foregoing.

In a feature of this aspect of the invention, the anesthetic solution vehicle may include an aqueous form.

In another feature of this aspect of the invention, the anesthetic solution vehicle may include a non-aqueous form.

In another feature of this aspect of the invention, the anesthetic agent may be selected from the group consisting of: lidocaine derivates; tetracaine derivates; xylocaine derivates; mepivacaine derivates; prilocaine derivates; bupivacaine derivates; etidocaine derivates; ropivacaine derivates; articaine derivates; and combinations of any of the foregoing. In a variation of this feature, the anesthetic agent may be present in the solution in a mass percentage that ranges from about 0.1% to about 10%.

In another feature of this aspect of the invention, the sugar may be selected from the group consisting of: dextrose; fructose; galactose; glyceraldehydes; lactose; maltose; sucrose; starch; glycogen; and combinations of any of the foregoing. In another feature of this aspect of the invention, the sugar may be present in the solution in amounts ranging from about 0.0001 to about 1,000 milligram per milliliter.

In another feature of this aspect of the invention, the bitterness suppressant may include a sweet compound selected from the group consisting of: stevia; aspartame; sucralose; neotame; acesulfame potassium; saccharin; sodium saccharin; advantame; cyclamates; lyzozyme, alitame; lactisole; corn syrup; high fructose corn syrup; sugar alcohols; thaumatin; glycosides; terpenoid glycosides; polyhydric alcohols; dipeptide derivatives; N-sulfonylamides; sulfamates; proteins; dihydrochalcones; chlorodeoxysugars; polychlorodeoxysugars; monodeoxysugars; polydeoxysugars; and combinations of any of the foregoing.

In another feature of this aspect of the invention, the bitterness suppressant may include an acid selected from the group consisting of: acetic acid; lactic acid; malic acid; citric acid; and combinations of any of the foregoing.

In another feature of this aspect of the invention, the bitterness suppressant may include an amino acid selected from the group consisting of: lysine; glutamic acid; leucine; arginine; alanine; valine; isoleucine; aspartic acid; phenylalanine; glycine; serine; histidine; threonine; ornithine monohydrochloride; proline; methionine; tryptophan; cysteine; taurine; tyrosine; amino sugars; and combinations of any of the foregoing.

In another feature of this aspect of the invention, the bitterness suppressant may include a salt selected from the group consisting of: sodium chloride; potassium chloride; calcium chloride; magnesium chloride; sodium lactate; and combinations of any of the foregoing.

In another feature of this aspect of the invention, the bitterness suppressant may include a miscellaneous suppressant substance selected from the group consisting of: non-mint and non-citrus artificial flavorings; flavor enhancers; and combinations of any of the foregoing. In a variation of this feature, the miscellaneous suppressant substance may include an artificial flavoring selected from the group consisting of: buttery flavoring (diacetyl); banana flavoring (isoamyl acetate); bitter almond flavoring (benzaldehyde); cinnamon flavoring (cinnamaldehyde); fruity flavoring (ethyl propionate); grape flavoring (methyl anthranilate); pear flavoring (ethyl decadienoate); cotton candy flavoring (ethyl maltol); vanilla flavoring (ethylvanillin); and combinations of any of the foregoing. In another variation of this feature, the miscellaneous suppressant substance may include a flavor enhancer selected from the group consisting of: glutamic acid; glycine salts; guanylic acid salts; inosinic acid salts; 5-ribonucleotide salts; and combinations of any of the foregoing.

In another feature of this aspect of the invention, the improved local anesthetic solution may further include one or more additional agents selected from the group consisting of: buffering agents; vasoconstrictors; preservative compounds; stabilizers; contrast media agents; and combinations of any of the foregoing.

In another feature of this aspect of the invention, the additional agent may include a buffering agent selected from the group consisting of: one of the combinations of alkaline, neutral or acid substances of sodium bicarbonate; Hartmann's solution; Ringer's solution; lactated Ringer's solution; acetated Ringer's solution; bicarbonated Ringer's solution; colloids based agents; and combinations of the foregoing.

In another feature of this aspect of the invention, the additional agent may include a vasoconstrictor selected from the group consisting of: adrenaline; epinephrine; norepinephrine; phynylephrine; felypressin; levonordefrin; and combinations of any of the foregoing. In a variation of this feature, the vasoconstrictor may be present in a concentration ranging from about 1:10,000 to about 1:500,000 milligrams of vasoconstrictor per milliliter of solution

In another feature of this aspect of the invention, the additional agent may include a preservative compound selected from the group consisting of: paraben derivatives; sodium bisulfites or metabisulfites; ascorbic acid; benzyl alcohol; phenylethyl alcohol; phenol; meta-cresol; chlorobutanol; thimerosal; phenylmercuric salts; and combinations of any of the foregoing.

In another feature of this aspect of the invention, the additional agent may include a stabilizer selected from the group consisting of: tartaric acid; hydrochloric acid; citric acid; sodium; sodium hydroxide; triethanolamine; tromethamine; lecithin; fatty acid emulsions; polysorbate 80; and combinations of any of the foregoing.

In another feature of this aspect of the invention, the additional agent may include a contrast media agent selected from the group consisting of: silver-based agents; barium-based agents; ionic iodine; non-ionic iodine; sodium iodide, sodium iothalamate, amino sugar derivates; amino sugar derivates combined with iodinated compounds; gadolinium-based agents; iron; iron oxide; iron platinum; manganese; perflubron; nitrogen;

perfluorocarbon; protein-based and pharmaceutical prepared microbubble contrast media; and combinations of any of the foregoing.

In other features of this aspect of the invention, the solution may be contained within an anesthetic vial with a capacity ranging from about 0.3 milliliters to about 5 milliliters; and/or the solution may be administered with a dental syringe, a nasal spray, a needle-free injection system, or a hydrogel method.

Another aspect of the present invention relates to an improved local anesthetic solution with diminished bitter taste. An exemplary such local anesthetic solution includes an anesthetic agent, an anesthetic solution vehicle, a bitterness suppressant, and a buffering agent. Furthermore, in this aspect of the invention, the bitterness suppressant includes one or more compounds selected from the group consisting of: a sugar selected from the group consisting of monosaccharide sugars, disaccharide sugars, polysaccharide sugars, and combinations of the any of the foregoing; sweet-tasting compounds; acids; amino acids; salts; miscellaneous suppressant substances; and combinations of any of the foregoing.

In a feature of this aspect of the invention, the bitterness suppressant may include a sugar, and the buffering agent may include a lactated Ringer's solution.

In another feature of this aspect of the invention, the improved local anesthetic solution may further include a vasoconstrictor, wherein the vasoconstrictor includes epinephrine.

Another aspect of the present invention relates to an improved local anesthetic solution with diminished bitter taste. An exemplary such local anesthetic solution includes an anesthetic agent, an anesthetic solution vehicle, a bitterness suppressant, and a contrast media agent for enabling visualization of the solution using x-ray technology. Furthermore, in this aspect of the invention, the bitterness suppressant includes one or more compounds selected from the group consisting of: a sugar selected from the group consisting of monosaccharide sugars, disaccharide sugars, polysaccharide sugars, and combinations of the any of the foregoing; sweet-tasting compounds; acids; amino acids; salts; miscellaneous suppressant substances; and combinations of any of the foregoing.

In a feature of this aspect of the invention, the contrast media agent may include sodium iodide.

Another aspect of the present invention relates to a method of administering an improved local anesthetic solution with diminished bitter taste. An exemplary such method includes: (a) preparing the improved local anesthetic solution by adding an anesthetic agent, an anesthetic solution vehicle, and a bitterness suppressant to a vial, wherein the bitterness suppressant includes one or more compounds selected from the group consisting of: a sugar selected from the group consisting of monosaccharide sugars, disaccharide sugars, polysaccharide sugars, and combinations of the any of the foregoing; sweet-tasting compounds; acids; amino acids; salts; miscellaneous suppressant substances; and combinations of any of the foregoing; (b) loading the vial into an anesthesia administration device selected from the group consisting of: a dental syringe; a nasal spray; a needle-free injection system; and a hydrogel method; and (c) administering the anesthesia administration device to a circumscribed area for treatment.

In a feature of this aspect of the invention, the improved local anesthetic solution may further include one or more additional agents selected from the group consisting of: buffering agents; vasoconstrictors; preservative compounds; stabilizers; contrast media agents; and combinations of any of the foregoing.

In other features of this aspect of the invention, the circumscribed area may include a patient's nose; and/or the circumscribed area may include a patient's oral cavity accessed through nerve block or infiltration techniques.

In another feature of this aspect of the invention, the improved anesthetic solution may be applied through a dental syringe having a needle into a location selected from the group consisting of: the root canal system within a tooth; tooth surfaces for caries detection; tooth surfaces for crack and tooth fractures evaluation; periodontal sulcus for pocket measurement assessment; and dental implant templates.

In another feature of this aspect of the invention, the improved anesthetic solution may include a contrast media agent selected from the group consisting of: silver-based agents; barium-based agents; ionic iodine; non-ionic iodine; sodium iodide, sodium iothalamate, amino sugar derivates; amino sugar derivates combined with iodinated compounds; gadolinium-based agents; iron; iron oxide; iron platinum; manganese; perflubron; nitrogen; perfluorocarbon; protein-based and pharmaceutical prepared microbubble contrast media; and combinations of any of the foregoing. In a variation of this feature, the method may further include obtaining an x-ray image after administering the anesthesia administration device to the circumscribed area for treatment. In another variation of this feature, the step of obtaining the x-ray image may include use of a technique selected from the group consisting of: periapical film and digital dental imaging; low-dose dental imaging; dental fluoroscopy; panoramic imaging; cephalometric imaging; and CBCT scan.

In addition to the aforementioned aspects and features of the present invention, it should be noted that the present invention further encompasses the various possible combinations and subcombinations of such aspects and features. Thus, for example, any aspect may be combined with an aforementioned feature in accordance with the present invention without requiring any other aspect or feature.

### BRIEF DESCRIPTION OF THE DRAWINGS

One or more preferred embodiments of the present invention now will be described in detail with reference to the accompanying drawings, wherein the same elements are referred to with the same reference numerals, and wherein,

FIG. 1 is a schematic illustration of a vial containing ingredients of one formulation of the improved local anesthetic solution in accordance with one or more aspects of the present invention.

FIG. 2 is a schematic illustration of another formulation of the improved local anesthetic solution in accordance with one or more aspects of the present invention, shown in combination with contemplated methods of delivery.

FIG. 3 is an illustration of the molecular structure of a basic formulation of the improved local anesthetic solution in accordance with one or more aspects of the present invention.

FIG. 4A is a schematic illustration of a vial containing ingredients of another formulation of the improved local anesthetic solution in accordance with one or more aspects of the present invention.

FIG. 4B is an illustration of a molecular structure based on and/or derived from the improved local anesthetic solution formulation illustrated in FIG. 4A.

FIG. 5A is a schematic illustration of a vial containing ingredients of another formulation of the improved local anesthetic solution in accordance with one or more aspects of the present invention.

FIG. 5B is an illustration of a molecular structure based on and/or derived from the improved local anesthetic solution formulation illustrated in FIG. 5A.

FIG. 6 is a schematic illustration of a method of delivering the improved local anesthetic solution in accordance with one or more aspects of the present invention, shown in the context of dental use.

FIG. 7 is a schematic illustration of a method of delivering the improved local anesthetic solution in accordance with one or more aspects of the present invention, shown in the context of dental and contrast media use.

### DETAILED DESCRIPTION

As a preliminary matter, it will readily be understood by one having ordinary skill in the relevant art (“Ordinary Artisan”) that the present invention has broad utility and application. As should be understood, any embodiment may incorporate only one or a plurality of the above-disclosed aspects of the invention and may further incorporate only one or a plurality of the above-disclosed features. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention in any claim of a patent issuing here from, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability of 35 U.S.C. 112, paragraph 6 or subsection (f), no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, one or more preferred embodiments of the present invention are next described. The following description of one or more preferred embodiments is merely exemplary in nature and is in no way intended to limit the invention, its implementations, or uses.

Sweetness, which is generally acknowledged as being the opposite of bitterness, is one of five basic tastes and is universally regarded as a pleasurable experience. Common sweeteners used in the food and pharmaceutical industry include, but are not limited to, the following: lactose; maltose; sorbitol; glucose (dextrose); sucrose; fructose; galactose; glycosides; xylitol; corn syrup; high fructose corn syrup; sweet proteins, such as thaumatin; sweet amino acids, such as alanine, glycine and serine; antibiotic proteins, such as lysozyme; sweet inorganic compounds; artificial sweeteners, such as aspartame, sodium saccharin, acesulfame potassium, sucralose, alitame and neotame; sweetener modifiers, such as lactisole; and artificial flavors or their byproducts as a result of industrial synthesis or biosynthesis. Sweetness appears to have one of the highest taste recognition thresholds and is generally capable of detection at about 1 part in 200 of sucrose in solution.

Sucrose, which is a common example of a sweet substance, has a sweetness perception rating of 1 when dissolved in solution. Other sweet substances are rated relative to this standard. For example, another common sugar, fructose, is somewhat sweeter than sucrose, and has a sweetness perception raking that is 1.7 times the sweetness of sucrose in solution. Sorbitol, a common sugar alcohol, has a sweetness perception raking that 0.6 times the sweetness of sucrose in solution. Sodas, such as COCA COLA® (manufactured by The Coca-Cola Company of Atlanta, GA), are generally equivalent in sweetness to a 0.33 M concentration of sucrose in solution.

By comparison with the above, bitterness appears to have one of the lowest detection thresholds—at about 1 part in 2 million for quinine in solution. Lidocaine, mepivacaine, articaine, and epinephrine are bitter agents commonly used as primary ingredients in dental anesthetic formulations.

An improved local anesthetic solution for dental use in accordance with one or more aspects of the present invention decreases the bitter taste common to anesthetic solutions for dental use. Decreasing the bitter taste is accomplished through the inclusion of a bitterness suppressant within the anesthetic solution vehicle in combination with one or more anesthetic agents. Improved formulations in accordance with one or more aspects of the present invention, as described herein, optionally include one or more additional agents, such as buffering agents, vasoconstrictors, preservative compounds, stabilizers, or one or more radiopaque substances (i.e., contrast media agents).

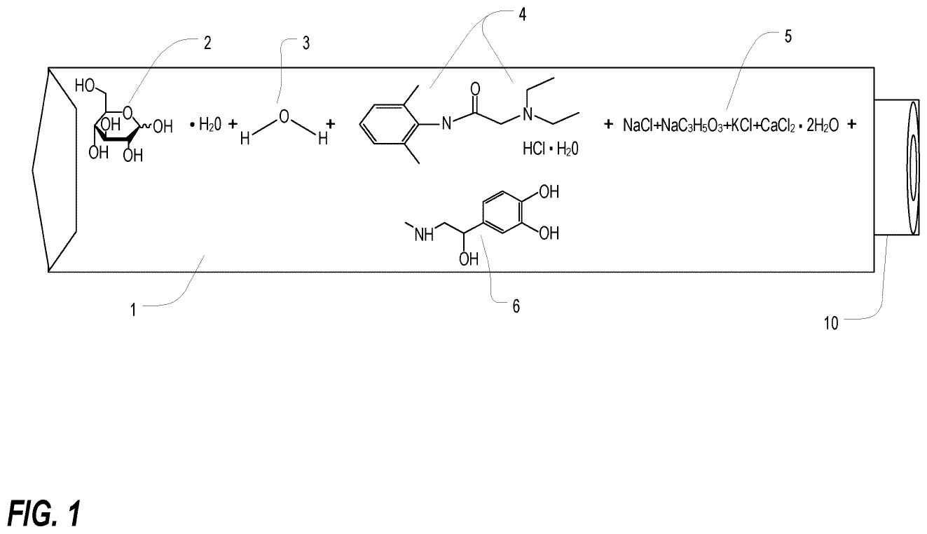

FIG. 1 is a schematic illustration of a vial 10 containing ingredients of one formulation of the improved local anesthetic solution 1 in accordance with one or more aspects of the present invention. As shown in FIG. 1, it is contemplated that ingredients of an improved local anesthetic solution 1 in accordance with one or more aspects of the present invention are contained within an anesthetic vial 10. Though a specific selection of ingredients is depicted in FIG. 1, such ingredients are illustrative of one contemplated formulation of the improved local anesthetic solution.

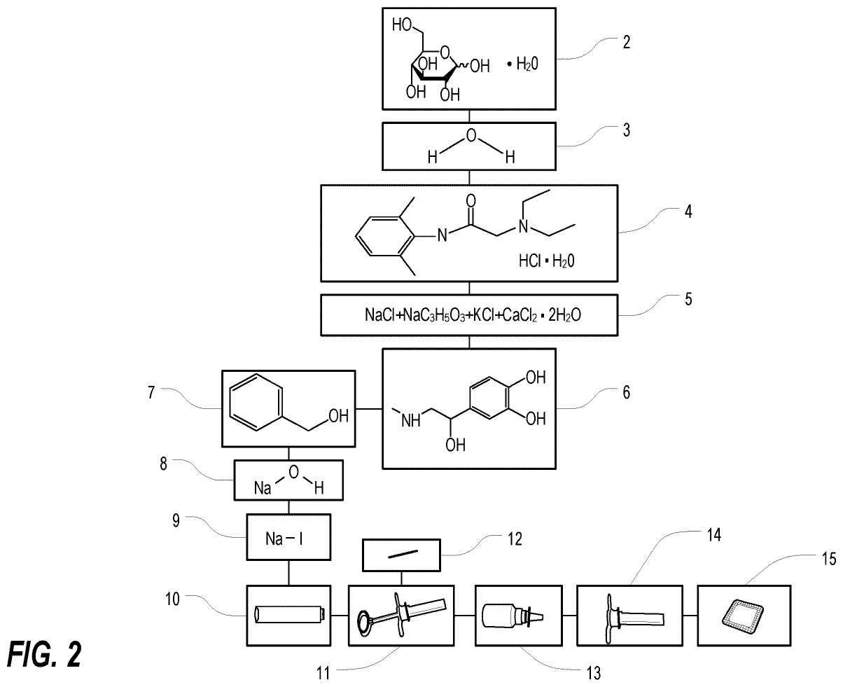

Once the anesthetic vial 10 is filled with the selected ingredients, the vial is ready for placement within a selected delivery mechanism. Possible delivery mechanisms include, but are not limited to, a dental syringe 11 with a needle 12, nasal sprays 13, and needle-free injection systems 14 (each of which is depicted in FIG. 2, discussed below). Furthermore, it is contemplated that the improved local anesthetic solution can be administered to the oral cavity, nose and circumscribed areas for dental use through the infiltration or nerve block methods.

FIG. 2 is a schematic illustration of another formulation of the improved local anesthetic solution in accordance with one or more aspects of the present invention, shown in combination with contemplated methods of delivery. The formulation of FIG. 2 is similar to that of FIG. 1, but is illustrated with the inclusion of additional components and in combination with contemplated methods of delivery. As shown generally in FIGS. 1 and 2, an improved local anesthetic solution 1 includes a bitterness suppressant 2 within an anesthetic solution vehicle 3 in combination with one or more anesthetic agents 4. Inclusion of the bitter suppressant 2 decreases the bitter taste of the anesthetic solution.

In various contemplated embodiments, the bitterness suppressant 2 includes one or more compounds selected from the following classes of compounds: sugars; sweet-tasting compounds; acids; amino acids; salts; miscellaneous suppressant substances; and combinations of any of the foregoing.

Bitterness suppressant sugars for use in accordance with an improved local anesthetic solution for dental use as described herein are generally made from one or a combination of two or more monosaccharides, disaccharides or polysaccharides, in amounts ranging from 0.0001 to 1,000 mg per milliliter. Contemplated saccharides include, but are not limited to: dextrose; fructose; galactose; glyceraldehydes; lactose; maltose; sucrose; starch; glycogen; and combinations thereof. Bitterness suppressant sweet-tasting compounds include one or more compounds from the group that includes, but is not limited to: stevia; aspartame; sucralose; neotame; acesulfame potassium; saccharin; sodium saccharin; advantame; cyclamates; lyzozyme, alitame; lactisole; corn syrup; high fructose corn syrup; sugar alcohols; thaumatin; glycosides; terpenoid glycosides; polyhydric alcohols; dipeptide derivatives; N-sulfonylamides; sulfamates; proteins; dihydrochalcones; chlorodeoxysugars; polychlorodeoxysugars; monodeoxysugars; and polydeoxysugars. Other sugars and sweet compounds are likewise contemplated.

Bitterness suppressant acids for use in accordance with an improved local anesthetic solution as described herein are generally made from one or more acids from the group that includes, but is not limited to: acetic acid; lactic acid; malic acid; and citric acid. Other similar acids are likewise contemplated.

Bitterness suppressant amino acids and/or L-form amino acids for use in accordance with an improved local anesthetic solution as described herein include one or more amino acids from the group that includes, but is not limited to: lysine; glutamic acid; leucine; arginine; alanine; valine; isoleucine; aspartic acid; phenylalanine; glycine; serine; histidine; threonine; ornithine monohydrochloride; proline; methionine; tryptophan; cysteine; taurine; and tyrosine. Amino sugars and other amino acids are likewise contemplated.

Bitterness suppressant salts for use in accordance with an improved local anesthetic solution as described herein are comprised of one or more salts from the group that includes, but is not limited to: sodium chloride; potassium chloride; calcium chloride; magnesium chloride; and sodium lactate. Other similar salts used intravenously or through intradermal, intramuscular, subcutaneous and intramucosal routes are likewise contemplated.

Miscellaneous suppressant substances include one or more compounds from the group that includes, but is not limited to: non-mint and non-citrus artificial flavorings; and flavor enhancers. Contemplated artificial flavorings include, but are not limited to: buttery flavoring (diacetyl); banana flavoring (isoamyl acetate); bitter almond flavoring (benzaldehyde); cinnamon flavoring (cinnamaldehyde); fruity flavoring (ethyl propionate); grape flavoring (methyl anthranilate); pear flavoring (ethyl decadienoate); cotton candy flavoring (ethyl maltol); and vanilla flavoring (ethylvanillin). Contemplated flavor enhancers include, but are not limited to: glutamic acid; glycine salts; guanylic acid salts; inosinic acid salts; and 5-ribonucleotide salts. It is further contemplated that artificial flavorings of various selected flavors are combinable with one another. Other artificial flavorings, flavor enhancers, and variations thereof are likewise contemplated.

The anesthetic solution vehicle 3 for use in accordance with an improved local anesthetic solution for dental use as described herein may have an aqueous or a non-aqueous form. In contemplated embodiments, the anesthetic solution vehicle includes one or more solution vehicles from the group that includes, but is not limited to: water; hypotonic, isotonic or hypertonic crystalloids solutions; colloids solutions; hydrophilic polymer chains networks; and plasma.

The anesthetic agent 4 for use in accordance with an improved local anesthetic solution for dental use as described herein includes one or more agents from the group that includes, but is not limited to: lidocaine derivates; tetracaine derivates; xylocaine derivates; mepivacaine derivates; prilocaine derivates; bupivacaine derivates; etidocaine derivates; ropivacaine derivates; and articaine derivates, each in mass percentages that range from about 0.1% to about 10%. Other anesthetic agents and variations thereof are likewise contemplated.

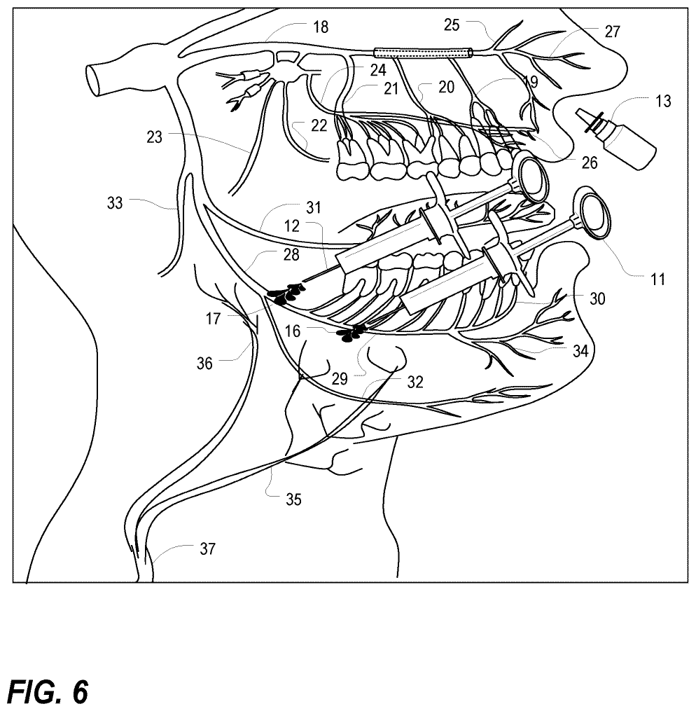

As further shown in FIGS. 1 and 2, an improved local anesthetic solution formulation as described herein may optionally include one or more additional agents, such as buffering agents 5, vasoconstrictors 6, preservative compounds 7, other stabilizers 8, and/or contrast media agents 9. The components illustrated herein are contained within an anesthetic vial 10 to be placed in a dental syringe 11 with a needle 12, nasal sprays anesthetic bottles 13, needle-free injection systems 14 or by other methods 15 (i.e., a hydrogel method) capable of administering the improved local anesthetic solution in the oral cavity, nose and circumscribed areas through the infiltration 16 or nerve block 17 methods (discussed in connection with FIG. 6).

Buffering agents 5 generally include a solution of one or more bases or alkali salts, such as sodium bicarbonate, and are often utilized to help prevent rapid adjustment of the pH of a solution. Sodium bicarbonate is incompatible with epinephrine. Additionally, sodium bicarbonate, a weak base, may react in the presence of water with weak acids used as stabilizers, such as hydrochloric acid. This reaction would release carbon dioxide, which makes the solution contained within the anesthetic vial 10 instable. In a contemplated embodiment, a lactated Ringer's solution is utilized to buffer the sugar-based bitterness suppressant.

Ringer's saline solution of inorganic salts was invented in the early 1880s by Sydney Ringer, a British physician and physiologist. Years later, the original Ringer's solution was further modified by American pediatrician Alexis Hartmann for the purpose of treating acidosis. Hartmann added lactate, which mitigates changes in pH by acting as a buffer for acid.

A lactated Ringer's solution is a solution that is generally isotonic with blood and commonly used for intravenous administration. It is also commonly administered subcutaneously. Lactated Ringer's solution is grouped with intravenous fluids known as crystalloids, which include saline and dextrose solutions. Lactated Ringer's solution has an osmolarity of 273 mOsm/L. The lactate is metabolized into bicarbonate by the liver, which can help correct metabolic acidosis. Advantageously, lactated Ringer's solution and its variants are safe for use in the human body.

Buffering agents 5 for use in connection with an improved local anesthetic solution in accordance with one or more aspects of the present invention include one or more agents from the group that includes, but is not limited to: one of the combinations of alkaline, neutral or acid substances of sodium bicarbonate; Hartmann's solution; Ringer's solution; lactated Ringer's solution; acetated Ringer's solution; bicarbonated Ringer's solution; and colloids based agents. Other buffering agents and variations thereof are likewise contemplated.

One or more vasoconstrictors 6 may be implemented into the solution to help constrict blood vessels during use of the anesthetic solution, and thereby reduce the risk of localized bleeding. Vasoconstrictors 6 for use in connection with an improved local anesthetic solution in accordance with one or more aspects of the present invention include one or more agents from the group that includes, but is not limited to: adrenaline; epinephrine; norepinephrine; phynylephrine; felypressin; and levonordefrin. Concentrations of the vasoconstrictor 6 range from about 1:10,000 to about 1:500,000 (mg of vasoconstrictor per ml of solution). Other vasoconstrictors and variations thereof are likewise contemplated.

One or more preservative compounds 7 may optionally be incorporated into the solution to preserve the solution against decay over an extended period of time. Preservative compounds 7 for use in connection with an improved local anesthetic solution in accordance with one or more aspects of the present invention include one or more agents from the group that includes, but is not limited to: paraben derivatives; sodium bisulfites or metabisulfites; ascorbic acid; benzyl alcohol; phenylethyl alcohol; phenol; meta-cresol; chlorobutanol; thimerosal; and phenylmercuric salts. Other preservative compounds are likewise contemplated.

One or more stabilizers 8 may optionally be included in the solution to stabilize the solution and inhibit unnecessary reactions from occurring. Stabilizers 8 for use in connection with an improved local anesthetic solution in accordance with one or more aspects of the present invention include one or more agents from the group that includes, but is not limited to: tartaric acid, hydrochloric acid, citric acid, sodium, sodium hydroxide, triethanolamine, tromethamine, lecithin, fatty acid emulsions and polysorbate 80. Other stabilizers and similar chemical additions are likewise contemplated.

It is further contemplated that one or more contrast media agents 9 may optionally be included in the solution to make the solution visible using x-ray technology. Contrast media agents 9 for use in connection with an improved local anesthetic solution in accordance with one or more aspects of the present invention include one or more agents from the group that includes, but is not limited to: silver-based agents; barium-based agents; ionic iodine; non-ionic iodine; sodium iodide; sodium iothalamate and other salts; amino sugar derivates and their combinations with iodinated compounds; gadolinium-based agents; iron; iron oxide; iron platinum; manganese; perflubron; nitrogen; perfluorocarbon; and protein-based and pharmaceutical prepared microbubble contrast media. Other contrast media agents are likewise contemplated.

The bitter taste of common anesthesia solutions is suppressable by the inclusion of a bitterness suppressant 2 and, optionally, one or more additional agents as discussed above. In one contemplated mode of operation, bitter taste is suppressed by the inclusion of a sweet-tasting substance (i.e., a sugar or other sweet-tasting compound), which operates to overwhelm the inherent bitter taste. In another contemplated mode of operation, bitter taste is suppressed by adjustment of the pH buffer in solution. For example, an acidic solution can be adjusted by the inclusion of a base or an alkali salt to neutralize the pH. In various contemplated embodiments, either or both modes of operation are involved in suppressing the bitter taste of a given anesthetic solution.

Additionally, components used in connection with the improved local anesthetic solution are understood to be safe for use in the human body, even when combined together. For instance, it has been shown that dextrose, also known as ‘blood sugar’, in combination with vasoconstrictors, has been safely used in patients for spinal anesthesia.

The components described herein are mixed and contained within an anesthetic vial 10 (as shown in FIGS. 1 and 2) with a capacity ranging from 0.3 ml to 5 ml. The anesthetic vial 10 can be placed in a dental syringe 11 made of stainless steel, disposable plastic or other material. Contemplated syringes include a conventional dental syringe, an intraligamentary syringe or gun, and an intraosseous system, with a needle 12 having a 10 to 40 gauge range and different sizes from 5-40 mm. Nasal spray anesthetics bottles 13 include local decongestants along with the anesthetic agents 4 and the bitterness suppressant 2 as described herein. Other administration methods, such as a needle-free injection system 14, stand to benefit from this formulation. It is further contemplated that an improved local anesthetic solution in accordance with one or more aspects of the present invention can be administered by other methods 15, such as the gel-forming material and Bukofitom hydrogel method, which is capable of administering the anesthetic solution in the oral cavity, nose and circumscribed areas on a local anesthetic approach through the infiltration 16 or nerve block 17 methods (discussed in connection with FIG. 6).

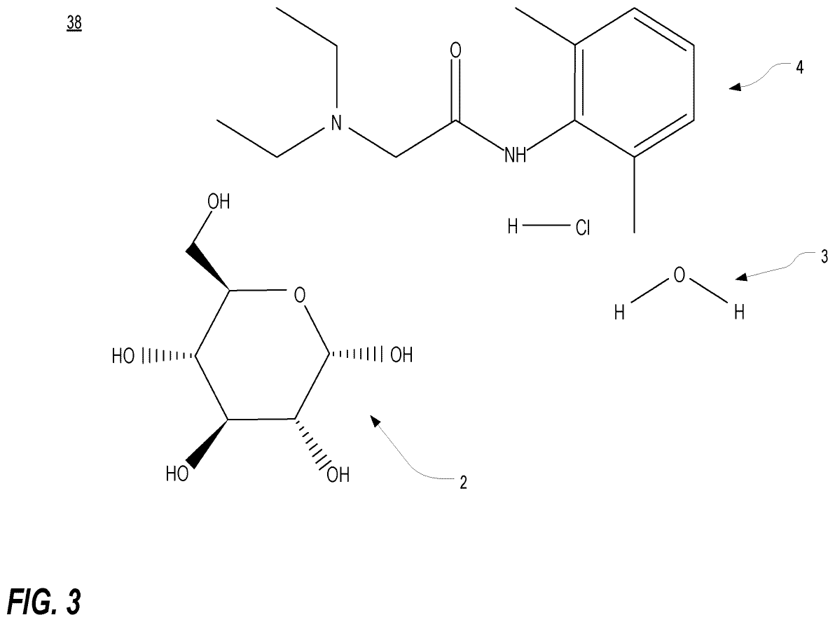

FIG. 3 is an illustration of the molecular structure of a basic formulation of the improved local anesthetic solution in accordance with one or more aspects of the present invention. In FIG. 3, an improved local anesthetic solution basic formulation 38 includes a bitterness suppressant 2 (shown as dextrose), an anesthetic solution vehicle 3 (shown as water), and an anesthetic agent 4 (shown as lidocaine hydrochloride). The chemical formula of the improved local anesthetic solution basic formulation 38 is C20H37ClN2O8 with an exact mass of 468.22 and a molecular weight of 468.97. Its elemental analysis is shown below in Table 1.

TABLE 1
Elemental Analysis of FIG. 3 Formulation

| Element | Weight % |
|---|---|
| Carbon (C) | 51.22 |
| Hydrogen (H) | 7.95 |
| Chlorine (Cl) | 7.56 |
| Nitrogen (N) | 5.97 |
| Oxygen (O) | 27.29 |

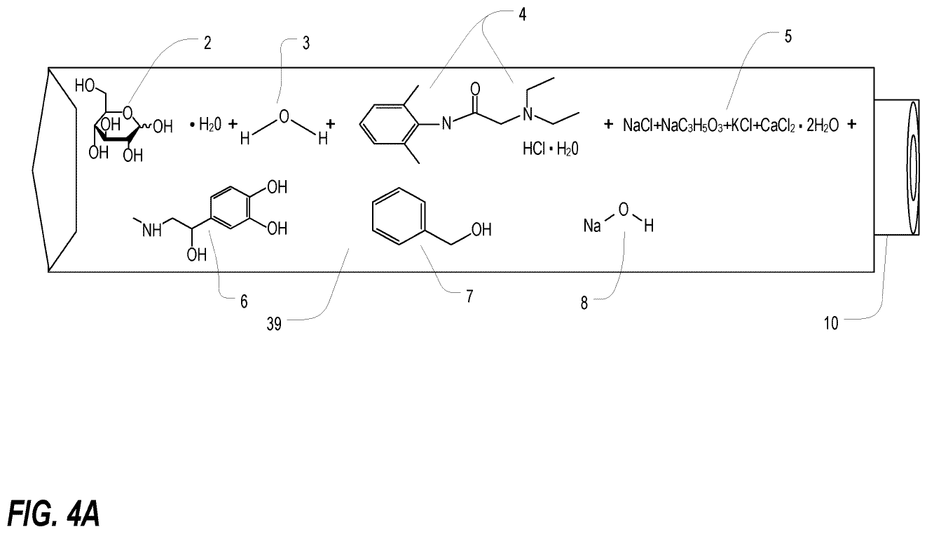

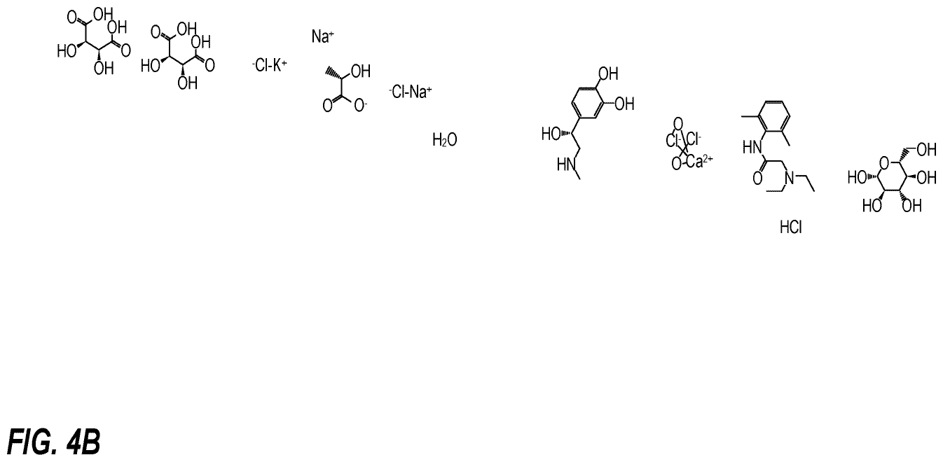

FIG. 4A is a schematic illustration of a vial containing ingredients of another formulation of the improved local anesthetic solution in accordance with one or more aspects of the present invention. As shown in FIG. 4A, the improved local anesthetic solution basic formulation 38 is compounded through the addition of epinephrine and a lactated Ringer's solution to form a compounded local anesthetic solution formulation 39. The compounded local anesthetic solution formulation 39 includes a bitterness suppressant 2 (shown as dextrose), an anesthetic solution vehicle 3 (shown as water), an anesthetic agent 4 (shown as lidocaine hydrochloride), a buffering agent 5 (shown as lactated Ringer's solution), a vasoconstrictor 6 (shown as epinephrine), a preservative compound 7 (shown as benzyl alcohol), and a stabilizer 8 (shown as sodium hydroxide).

FIG. 4B is an illustration of a molecular structure based on and/or derived from the improved local anesthetic solution formulation illustrated in FIG. 4A. The chemical formula of the molecular structure shown in FIG. 4B is understood to be C40H67CaCl5KN3Na2O28, with an exact mass of 1337.14 g and a molecular weight of 1340.37 g/mol. The elemental analysis of this molecular structure is shown in Table 2.

TABLE 2
Elemental Analysis of FIG. 4B Molecular Structure

| Element | Weight % |
|---|---|
| Carbon (C) | 35.84 |
| Hydrogen (H) | 5.04 |
| Calcium (Ca) | 2.99 |
| Chlorine (Cl) | 13.22 |
| Potassium (K) | 2.92 |
| Nitrogen (N) | 3.14 |
| Sodium (Na) | 3.43 |
| Oxygen (O) | 33.42 |

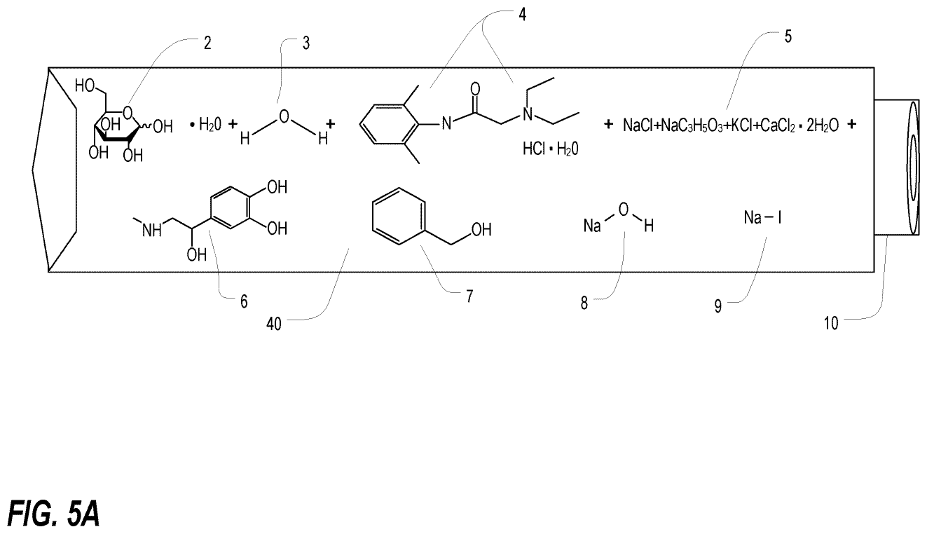

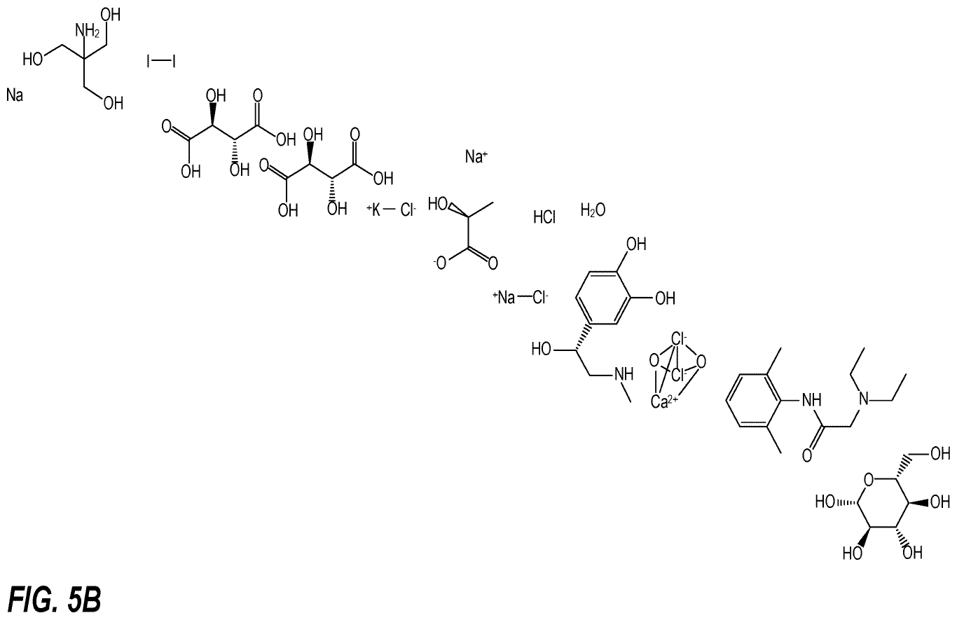

FIG. 5A is a schematic illustration of a vial containing ingredients of another formulation of the improved local anesthetic solution in accordance with one or more aspects of the present invention. As shown in FIG. 5A, the compounded local anesthetic solution formulation 39 of FIG. 4A is modified for use with contrast media with the inclusion of sodium iodide to form a compounded local anesthetic solution formulation 40 for dental and contrast media use. The improved local anesthetic solution formulation 40 for dental and contrast media use includes a bitterness suppressant 2 (shown as dextrose), an anesthetic solution vehicle 3 (shown as water), an anesthetic agent 4 (shown as lidocaine hydrochloride), a buffering agent 5 (shown as lactated Ringer's solution), a vasoconstrictor 6 (shown as epinephrine), a preservative compound 7 (shown as benzyl alcohol), a stabilizer 8 (shown as sodium hydroxide), and a contrast media agent 9 (shown as sodium iodide).

FIG. 5B is an illustration of a molecular structure based on and/or derived from the improved local anesthetic solution formulation illustrated in FIG. 5A. The chemical formula of the molecular structure shown n FIG. 5B is understood to be C44H78CaCl512KN4Na3O31, with an exact mass of 1735.01 g and a molecular weight of 1738.31 g/mol. The elemental analysis of this molecular structure is shown in Table 3.

TABLE 3
Elemental Analysis of FIG. 5B Molecular Structure

| Element | Weight % |
|---|---|
| Carbon (C) | 30.40 |
| Hydrogen (H) | 4.52 |
| Calcium (Ca) | 2.31 |
| Chlorine (Cl) | 10.20 |
| Iodine (I) | 14.60 |
| Potassium (K) | 2.25 |
| Nitrogen (N) | 3.22 |
| Sodium (Na) | 3.97 |
| Oxygen (O) | 28.53 |

The improved local anesthetic solution basic formulation 38, the compounded local anesthetic solution formulation 39, and the improved local anesthetic solution formulation 40 for dental and contrast media use described herein may optionally include one or more additional agents, such as buffering agents 5, vasoconstrictors 6, preservative compounds 7 and other stabilizers 8.

FIG. 6 is a schematic illustration of a method of delivering an improved local anesthetic solution in accordance with one or more aspects of the present invention, shown in the context of dental use. With reference to FIGS. 2 and 6, the infiltration method 16 for administering an improved local anesthetic solution at the maxillary nerve 18 (2^{nd} division of the trigeminal nerve) level aims to anesthetize the anterior superior alveolar 19, middle superior alveolar 20, posterior superior alveolar 21, greater palatine 22, lesser palatine 23, nasopalatine 24, infra-orbital 25, labial superior 26, and nasal nerves 27 upon diffusion of the formulation. Administration of the improved local anesthetic solution is completed via a dental syringe 11 with a needle 12, nasal spray anesthetics 13, a needle-free injection system 14, or by other methods 15 capable of positioning the improved local anesthetic solution as close as possible to the desired area to be anesthetized.

The infiltration method 16 for administering an improved local anesthetic solution at the mandibular nerve 28 (3^{rd} division of the trigeminal nerve) involves buccal infiltration of the inferior alveolar nerve 29 (in children) or the incisive nerve 30 (in children and adults) as well as intraligametary and intraosseous techniques. Administration of the improved local anesthetic solution is completed via a dental syringe 11 with a needle 12, needle-free injection systems 14, or by other methods 15 capable of positioning the improved local anesthetic solution as close as possible to the desired area to be anesthetized.

For the nerve block 17 of the inferior alveolar 29, lingual 31, mylohyoid 32, long buccal 33 and mental nerve 34, administration of an improved local anesthetic solution is completed via a syringe 11 with a needle 12, needle-free injection systems 14, or by other methods 15 suitable to position the improved local anesthetic solution near the nerve to be anesthetized.

For the infiltration method 16 aiming to anesthetize the transverse cervical 35 and greater auricular 36 nerves from the cervical plexus 37 branch, a syringe 11 with a needle 12, needle-free injection systems 14, or other methods 15 can be utilized in order to administer an improved local anesthetic solution near the area to be anesthetized.

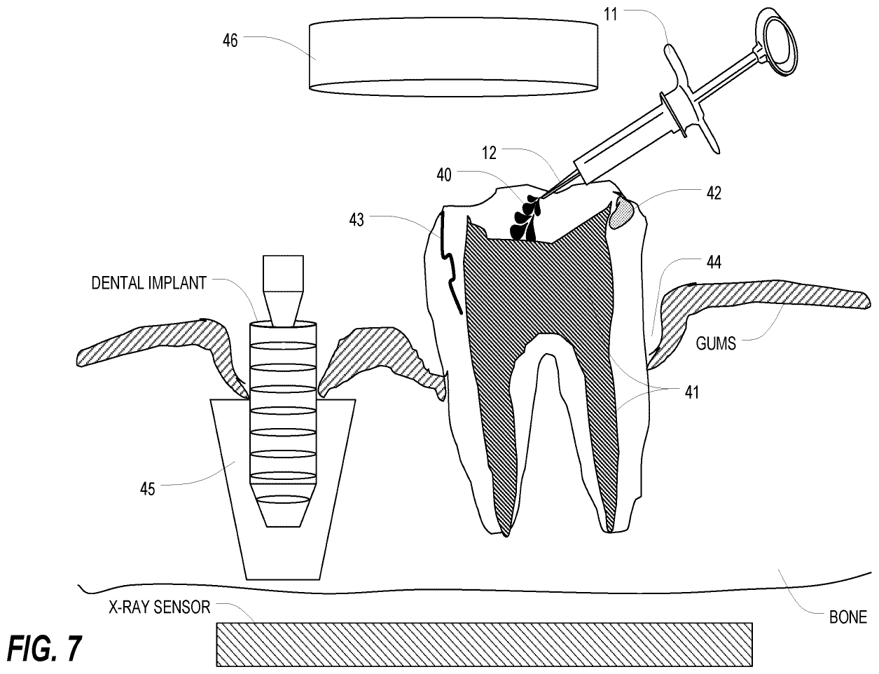

FIG. 7 is a schematic illustration of a method of delivering the improved local anesthetic solution in accordance with one or more aspects of the present invention, shown in the context of dental and contrast media use. As shown in FIG. 7, the improved local anesthetic solution for dental and contrast media use 40 method can be applied through a syringe 11 and a needle 12 into the root canal system 41 within a tooth, tooth surfaces for caries detection 42, tooth surfaces for crack and tooth fractures 43 evaluation, periodontal sulcus for pocket measurement 44 assessment and dental implant templates 45 in which the provisional restoration, the blueprint for the final restoration, is duplicated in a radiopaque radiographic template. The method described herein includes x-ray imaging 46 after the improved local anesthetic solution is administered for dental and contrast media use 40 applications in the desired head and neck area. X-ray imaging 46 includes one or more of the following techniques that include, but are not limited to: conventional periapical film and digital dental imaging; low-dose dental imaging; dental fluoroscopy; panoramic imaging; cephalometric imaging; and CBCT scan.

### EXAMPLES

The examples described herein set forth a variety of improved local anesthetic solution formulations made in accordance with one or more aspects of the present invention. Selection of ingredients and amounts thereof may be varied to be suitable for a particular set of use circumstances. For example, selection and amounts of ingredients such as citric acid, sodium hydroxide, and metabisulfites may be varied to accommodate for variations in the solution pH.

Example 1: In a first example of the improved local anesthetic solution for dental use in accordance with one or more aspects of the present invention, a 2% lidocaine in 5% dextrose lactated Ringer's solution contained in a 1.7 ml vial includes: a 1.7 ml water solution of 34 mg lidocaine hydrochloride monohydrate; 85 mg of dextrose monohydrate; 10.2 mg of sodium chloride; 5.27 mg of sodium lactate; 0.51 mg of potassium chloride; 0.34 mg of calcium chloride; 0.85 mg of sodium hydroxide; 0.34 mg of citric acid; and 0.85 mg of sodium metabisulfite.

Example 2: In a second example of the improved local anesthetic solution for dental use in accordance with one or more aspects of the present invention, a 2% lidocaine in 10% dextrose lactated Ringer's solution contained in a 1.7 ml vial includes: a 1.7 ml water solution of 34 mg lidocaine hydrochloride monohydrate; 170 mg of dextrose monohydrate; 10.2 mg of sodium chloride; 5.27 mg of sodium lactate; 0.51 mg of potassium chloride; 0.34 mg of calcium chloride; 0.85 mg of sodium hydroxide; 0.34 mg of citric acid; and 0.85 mg of sodium metabisulfite.

Example 3: In a third example of the improved local anesthetic solution for dental use in accordance with one or more aspects of the present invention, a 2% lidocaine in 5% dextrose lactated Ringer's solution with 1:100,000 epinephrine solution contained in a 1.7 ml vial includes: a 1.7 ml water solution of 34 mg lidocaine hydrochloride monohydrate; 85 mg of dextrose monohydrate; 10.2 mg of sodium chloride; 5.27 mg of sodium lactate; 0.51 mg of potassium chloride; 0.34 mg of calcium chloride; 0.017 mg of epinephrine; 0.85 mg of sodium hydroxide; 0.34 mg of citric acid; and 0.85 mg of sodium metabisulfite.

Example 4: In a fourth example of the improved local anesthetic solution for dental use in accordance with one or more aspects of the present invention, a 2% lidocaine in 5% dextrose lactated Ringer's solution with 1:50,000 epinephrine solution contained in a 1.7 ml vial includes: a 1.7 ml water solution of 34 mg lidocaine hydrochloride monohydrate; 85 mg of dextrose monohydrate; 10.2 mg of sodium chloride; 5.27 mg of sodium lactate; 0.51 mg of potassium chloride; 0.34 mg of calcium chloride; 0.034 mg of epinephrine; 0.85 mg of sodium hydroxide; 0.34 mg of citric acid; and 0.85 mg of sodium metabisulfite.

Example 5: In a fifth example of the improved local anesthetic solution for dental use in accordance with one or more aspects of the present invention, a 3% mepivacaine in 5% dextrose lactated Ringer's solution contained in a 1.7 ml vial includes: a 1.7 ml water solution of 51 mg mepivacaine hydrochloride; 85 mg of dextrose monohydrate; 10.2 mg of sodium chloride; 5.27 mg of sodium lactate; 0.51 mg of potassium chloride; 0.34 mg of calcium chloride; 0.85 mg of sodium hydroxide; 0.34 mg of citric acid; and 0.85 mg of sodium metabisulfite.

Example 6: In a sixth example of the improved local anesthetic solution for dental use in accordance with one or more aspects of the present invention, a 2% lidocaine in 5% dextrose lactated Ringer's solution with 30% organically bound iodine contrast media contained in a 1.7 ml vial includes: a 1.7 ml water solution of 34 mg lidocaine hydrochloride monohydrate; 85 mg of dextrose monohydrate; 10.2 mg of sodium chloride; 5.27 mg of sodium lactate; 0.51 mg of potassium chloride; 0.34 mg of calcium chloride; 0.85 mg of sodium hydroxide; 0.34 mg of citric acid; 0.85 mg of sodium metabisulfite; 510 mg of organically bound iodine; 0.073 mg of sodium; and 0.113 mg of tromethamine.

Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention. Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

1.-36. (canceled)

37. A pharmaceutical composition comprising:
an anesthetic present as a pharmaceutically acceptable salt selected from the group consisting of lidocaine, mepivacaine, articaine, prilocaine, or bupivacaine; and
an aqueous vehicle including a Ringer's-type buffer and a sugar,
wherein the pharmaceutical composition is free of epinephrine and has an osmolality from 500 to 700 mOsm/kg.

38. The pharmaceutical composition of claim 37, wherein the aqueous vehicle comprises a lactated Ringer's solution.

39. The pharmaceutical composition of claim 37, wherein the sugar comprises at least one of dextrose, sodium saccharin, and glycerin.

40. The pharmaceutical composition of claim 37, further comprising citric acid and sodium hydroxide in amounts effective to maintain the pharmaceutical composition at a preselected pH during storage and use.

41. The pharmaceutical composition of claim 37, further comprising one or more amino acids.

42. The pharmaceutical composition of claim 41, wherein the one or more amino acids are effective to modulate at least one of taste, buffering capacity, and osmolality of the pharmaceutical composition.

43. The pharmaceutical composition of claim 41, wherein the one or more amino acids are selected from the group consisting of lysine, glutamic acid, leucine, arginine, alanine, valine, isoleucine, aspartic acid, phenylalanine, glycine, serine, histidine, threonine, ornithine monohydrochloride, proline, methionine, tryptophan, cysteine, taurine, and tyrosine.

44. The pharmaceutical composition of claim 37, wherein the anesthetic comprises one or more of lidocaine hydrochloride, mepivacaine hydrochloride, articaine hydrochloride, prilocaine hydrochloride, or bupivacaine hydrochloride.

45. The pharmaceutical composition of claim 37, further comprising citric acid and sodium hydroxide to maintain a pH of about 6.0 to about 7.5.

46. The pharmaceutical composition of claim 37, further comprising an organically bound iodine radiographic contrast medium configured to enhance radiographic visualization of an injection field of the pharmaceutical composition.

47. A system comprising the pharmaceutical composition of claim 37 filled in a dental syringe of about 1.7 mL.

48. A method of providing local anesthesia for dental treatment comprising injecting the pharmaceutical composition of claim 37 into oral cavity tissue of a patient to provide at least one of (i) a reduced perceived injection sting for the patient relative to an isotonic anesthetic composition, (ii) a shorter time to onset of soft-tissue anesthesia for the patient relative to an isotonic anesthetic composition, or (iii) a comparable anesthetic duration for the patient relative to a non-buffered anesthetic formulation containing epinephrine.

49. The method of claim 48, wherein the injection is aimed at the anterior superior alveolar, middle superior alveolar, posterior superior alveolar, greater palatine, lesser palatine, nasopalatine, infraorbital, labial superior, nasal, inferior alveolar, lingual, mylohyoid, buccal, mental, incisive, greater auricular, or transverse cervical nerve, via an infiltration or a block.

50. The method of claim 48, wherein the pharmaceutical composition is injected from a unit-dose dental cartridge of about 1.7 mL.

51. The method of claim 48, wherein the pharmaceutical composition further includes an organically bound iodine radiographic contrast medium.

52. The method of claim 51, further comprising generating an x-ray image of an injection field where the pharmaceutical composition was injected.
